(19) United States
(12) Patent Application Publication
(10) Pub. No.: US 2019/0237201 A1
(43) Pub. Date: Aug. 1, 2019

(54) SYSTEMS AND METHODS FOR CLINICAL PLANNING AND RISK MANAGEMENT

(71) Applicant: MIRUS LLC, Atlanta, GA (US)

(72) Inventors: Jordan Bauman, Atlanta, GA (US); Pam Cowart, Atlanta, GA (US); Jay Yadav, Sandy Springs, GA (US); Angad Singh, Marietta, GA (US); Noah Roth, Atlanta, GA (US)

(21) Appl. No.: 16/339,218

(22) PCT Filed: Oct. 3, 2017
(86) PCT No.: PCT/US17/54968

§ 371 Date: Apr. 3, 2019

Related U.S. Application Data
(60) Provisional application No. 62/403,214, filed on Oct. 3, 2016.

(51) Int. Cl. G16H 50/70; G16H 50/20; G16H 50/30; G16H 50/50; G06F 9/54

(52) U.S. Cl. CPC G16H 50/70; G16H 50/20; G06F 9/547; G16H 50/50; G16H 50/30

(57) ABSTRACT

Systems and methods for clinical planning and risk management are described herein. An example method can include receiving, using an application program interface (API), clinical data from an electronic medical record, and using the clinical data, creating a risk based model for clinical planning or management. Another example method can include receiving a patient-specific parameter from a navigation system, a wearable device, a smart implant, or a surgical tool, and using the patient-specific parameter, creating or updating a risk based model for clinical planning or management. Another example method can include aggregating population based risk for a medical provider from a plurality of data sources, and displaying the population based risk on a display device of a computing device.

### CROSS-REFERENCE TO RELATED APPLICATIONS

This application claims the benefit of U.S. Provisional Patent Application No. 62/403,214, filed on Oct. 3, 2016, entitled “SYSTEMS AND METHODS FOR CLINICAL PLANNING AND RISK MANAGEMENT,” the disclosure of which is expressly incorporated herein by reference in its entirety.

### BACKGROUND

Medical providers are interested in analytical methods and tools that use clinical and procedural risk factors in decision making regarding course of treatment, surgical planning, post-surgical care and follow up.

### SUMMARY

Systems and methods for clinical planning and risk management are described herein. An exemplary method can include receiving, using an application program interface (API), clinical data from an electronic medical record, and creating a risk based model for clinical planning or management using the clinical data.

Another exemplary method can include receiving a patient-specific parameter from a navigation system, a wearable device, a smart implant, or a smart surgical tool, and using the patient-specific parameter, creating or updating a model for clinical planning or management. It should be understood that the navigation system, wearable device, smart implant, or smart surgical tool can be configured to record data and optionally transmit such data to a remote computing device over a network.

Alternatively or additionally, the methods can optionally further include generating a patient-specific risk metric using the risk based model. Optionally, the patient-specific risk metric can be a unique synthetic risk metric based on a plurality of risk factors. Optionally, the patient-specific risk metric can be a unique synthetic risk metric based on a customized set of risk factors (e.g., a set of risk factors customized for a particular patient). Optionally, the patient-specific risk metric can be a risk of readmission, complication, or revision.

Alternatively or additionally, the risk based model can represent a progression of a condition or risk over time. Optionally, the method can further include estimating an optimal time for an intervention based on the model.

Alternatively or additionally, in addition to clinical data, the patient-specific parameter can be at least one of force, orientation, position, temperature, wear, loosening, range of motion, or combinations thereof.

Alternatively or additionally, the methods can optionally further include displaying the model on a display device of a computing device.

Another exemplary method can include aggregating population based risk for a medical provider from a plurality of data sources, and displaying the population based risk on a display device of a computing device. The medical provider can be a single practitioner, a practice group, a clinic, a hospital, or a network of providers, for example.

Another example method described herein can include receiving, at a server, patient data associated with a plurality of patients over a network, and storing, in memory accessible by the server, the patient data. The method can also include receiving, at the server, a user-defined predictive outcome over the network, and creating, using the server, a dataset for predictive model generation from the patient data. The method can further include generating, using the server, a predictive model by analyzing the dataset based on the user-defined predictive outcome, and transmitting, from the server, display data over the network. The display data can represent the user-defined predictive outcome for a new patient.

In some implementations, the display data can be a binary outcome plotted as a function of a continuous variable.

In some implementations, the method can further include displaying, at a graphical user interface (GUI) of a client device, the display data representing the user-defined predictive outcome for the new patient.

In some implementations, the patient data is received at the server using an application program interface (API) configured to interface with respective electronic medical records (EMRs) associated with the plurality of patients.

In some implementations, the patient data is received at the server via respective applications running on respective client devices associated with the plurality of patients.

In some implementations, the patient data is received at the server via a navigation system, a wearable device, a smart implant, or a smart surgical tool.

In some implementations, the step of creating the dataset for predictive model generation from the patient data using the server includes creating and appending one or more output vectors to elements of the patient data.

In some implementations, the step of analyzing the dataset based on the user-defined predictive outcome includes performing a statistical analysis of the patient data.

In some implementations, the statistical analysis is at least one of a logistic regression, a linear regression, a proportional hazards regression, or a generalized linear model (GLM).

In some implementations, the method can further include receiving, at the server, an actual outcome associated with the new patient, and updating, using the server, the patient data to include the actual outcome associated with the new patient.

In some implementations, the method can further include regenerating, using the server, the predictive model.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Furthermore, the drawings describe herein are non-limiting and describe the conceptual concepts of the invention. Like reference numerals designate corresponding parts throughout the several views.

FIG. 1 illustrates an example computing environment for implementing clinical planning and risk management as described herein.

FIG. 2 illustrates an aspect of the example computing environment of FIG. 1.

FIG. 3 is a block diagram of an example computing device.

FIGS. 4A and 4B illustrate aggregating population based risk for a medical provider (e.g., a single practitioner, a practice group, a clinic, a hospital, a network of providers, etc.).

FIG. 5 illustrates forming a relationship between disease progression and predicting appropriate timing of surgery.

FIG. 6 illustrates obtaining data in an automated fashion and synthesis of the data creating a unique synthetic profile of the patient.

FIG. 7 illustrates inputting patient-specific parameters obtained during surgery using smart implants and/or surgical tools into a predictive model.

FIG. 8 illustrates post-operative assessment and risk assessment using patient-specific parameters obtained using smart implants and/or surgical tools.

FIGS. 9A-9E illustrate providing real-time information from patient to a medical provider. FIG. 9A shows patient activity monitoring (e.g., using a wearable device) in real-time. FIG. 9B shows patient range of motion monitoring (e.g., using a wearable device) in real-time. FIG. 9C shows home care assessment of pain. FIGS. 9D and 9E show home care assessment of joint function—knee in FIG. 9D and hip in FIG. 9E.

FIG. 10 is a table summarizing various data sources and uses described herein.

FIG. 11 is a flow chart illustrating example operations for patient-specific predictive modelling.

FIG. 12 is a graph illustrating follow up versus baseline Oswestry Disability Index (ODI) scores according to an example described herein.

FIG. 13 is another graph illustrating follow up versus baseline Oswestry Disability Index (ODI) scores according to an example described herein.

FIG. 14 is a table illustrating dataset generation according to an example described herein.

FIG. 15 is a table illustrating baseline predictor variable values for a new patient (Patient A) according to an example described herein.

FIG. 16 is a graph illustrating Patient A′s probability of meeting an MCID threshold (e.g., a binary outcome) as a function of Activity Rank (e.g., a continuous variable) according to an example described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for clinical planning and risk management, it will become evident to those skilled in the art that the implementations are not limited thereto.

Exemplary embodiments of the present invention that are shown in the figures are summarized below. It is to be understood, however, that there is no intention to limit the invention to the forms described within this application. One skilled in the art can recognize that there are numerous modifications, equivalents and alternative constructions that fall within the spirit and scope of the invention.

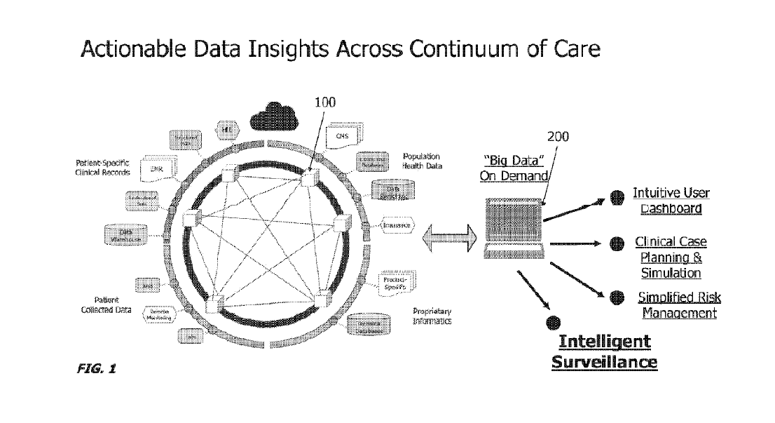

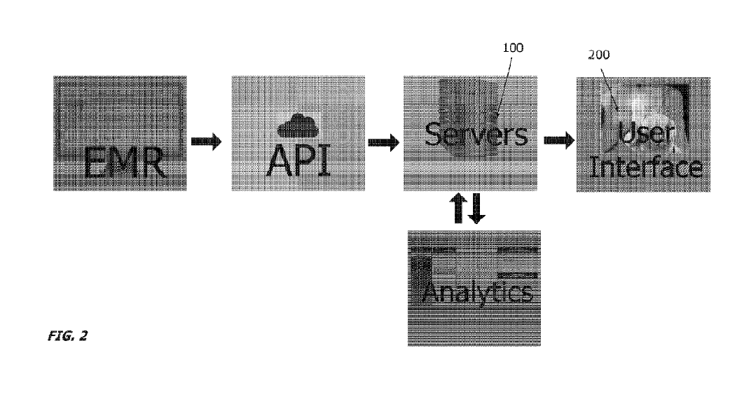

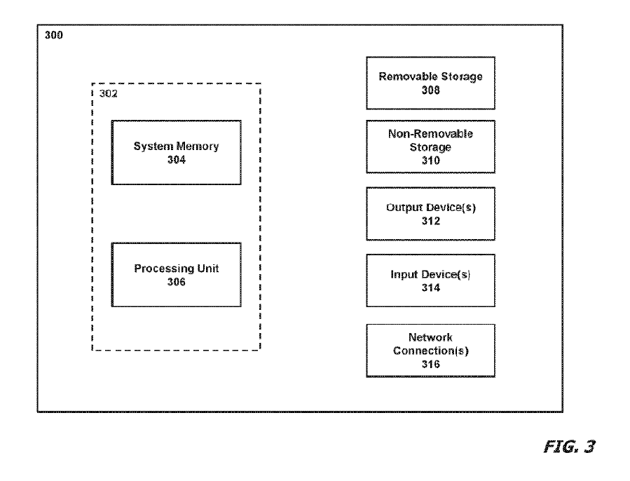

Referring now to FIGS. 1 and 2, an example computing environment for implementing techniques for clinical planning and risk management are shown. The computing environment can include one or more servers 100. The servers 100 can be connected by one or more networks. This disclosure contemplates that the networks are any suitable communication network. The networks can be similar to each other in one or more respects. Alternatively or additionally, the networks can be different from each other in one or more respects. The networks can include a local area network (LAN), a wireless local area network (WLAN), a wide area network (WAN), a metropolitan area network (MAN), a virtual private network (VPN), etc., including portions or combinations of any of the above networks. The servers 100 can be coupled to the networks through one or more communication links. This disclosure contemplates the communication links are any suitable communication link. For example, a communication link may be implemented by any medium that facilitates data exchange between the servers 100 including, but not limited to, wired, wireless and optical links. Example communication links include, but are not limited to, a LAN, a WAN, a MAN, Ethernet, the Internet, or any other wired or wireless link such as WiFi, WiMax, 3G or 4G. This disclosure contemplates that each server 100 can be a computing devices as described with regard to FIG. 3 (e.g., computing device 300). Optionally, the servers 100 can be implemented in a cloud computing environment. Cloud computing is well known in the art and is therefore not described in further detail herein.

The servers 100 can be configured to access and collect data from various sources (e.g., remote computing devices) over a network. For example, the servers 100 can collect data including, but not limited to, medical history, social history, comorbidities, demographic information, lab results, vital signs, wearable data, patient-reported outcomes, pain measures, functional measures, quality of life measures, and billing data. As shown in FIG. 1, the servers 100 can collect population health data, patient-specific clinical records, patient-collected data, and/or proprietary informatics. Optionally, the population health data, patient-specific clinical records, patient-collected data, and/or proprietary informatics can be stored by the servers 100. The population health data can include, but is not limited to, information obtained from Centers for Medicare and Medicaid Services (CMS) databases, clinical trial databases, insurance databases, or any other database. The patient-specific clinical data can include, but is not limited to, information obtained from electronic medical records (EMR) or other structured and unstructured clinical data. For example, patient-specific clinical data can be collected from EMRs using an application program interface (API) configured to provide access to and/or retrieve data from EMRs. This disclosure contemplates using any API known in the art for collecting clinical data from EMRs. The API facilitates the collection of patient-specific clinical data by the servers 100. This disclosure contemplates that clinical data includes any information regarding the diagnosis and/or treatment of a condition (e.g., disease, injury, disability, etc.) of a patient. The patient collected data can include, but is not limited to, results of laboratory tests, patient-provided data (e.g., pain scores, recovery metrics, etc.), patient activity monitoring data, patient-specific parameters measured using navigation systems, smart implants, and/or surgical tools (e.g., force, orientation, position, temperature, wear, range of motion, etc.). Examples of smart implants and/or surgical tools can be found in U.S. 2015/0297362, filed Nov. 1, 2013, titled “SYSTEMS AND METHODS FOR MEASURING ORTHOPEDIC PARAMETERS IN ARTHROPLASTIC PROCEDURES;” U.S. 2016/0007909, filed Sep. 21, 2015, titled “SYSTEMS AND METHODS FOR MEASURING PERFORMANCE PARAMETERS RELATED TO ORTHOPEDIC ARTHROPLASTY;” and WO 2015/196131, filed Jun. 19, 2015, titled “SYSTEMS AND METHODS FOR MEASURING PERFORMANCE PARAMETERS RELATED TO ARTIFICIAL ORTHOPEDIC JOINTS,” the disclosures of which are incorporated herein by reference in their entireties. Example navigation systems are described in WO 2017/151734, filed Mar. 1, 2017, titled “SYSTEMS AND METHODS FOR POSITION AND ORIENTATION TRACKING OF ANATOMY AND SURGICAL INSTRUMENTS,” the disclosure of which is incorporated herein by reference in its entirety.

The servers 100 can be communicatively connected to one or more client devices 200 over a network. As described above, this disclosure contemplates that the networks are any suitable communication network, and the servers 100 and client devices 200 can be coupled to the networks through one or more communication links, which can be any suitable communication link. Optionally, the client devices 200 can be a smart phone, tablet computer, laptop computer, desktop computer, or other computing device. For example, this disclosure contemplates that each client device 200 can be a computing devices as described with regard to FIG. 3 (e.g., computing device 300). The client devices 200 can include a display configured for displaying a user interface.

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which embodiments of the invention may be implemented is illustrated. It should be understood that the example computing device 300 is only one example of a suitable computing environment upon which embodiments of the invention may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

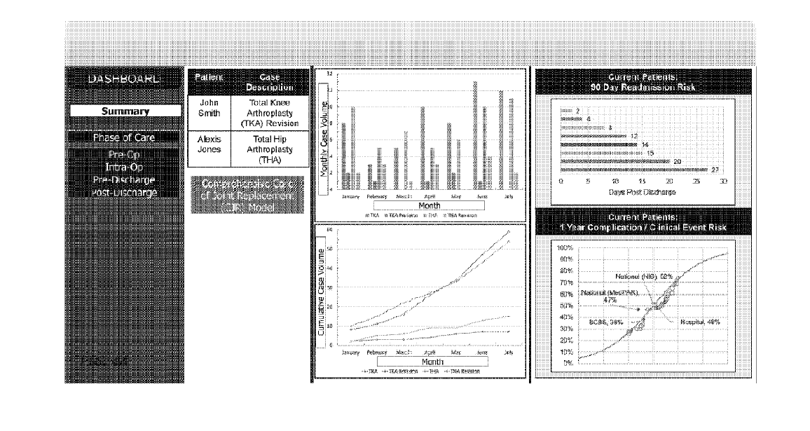

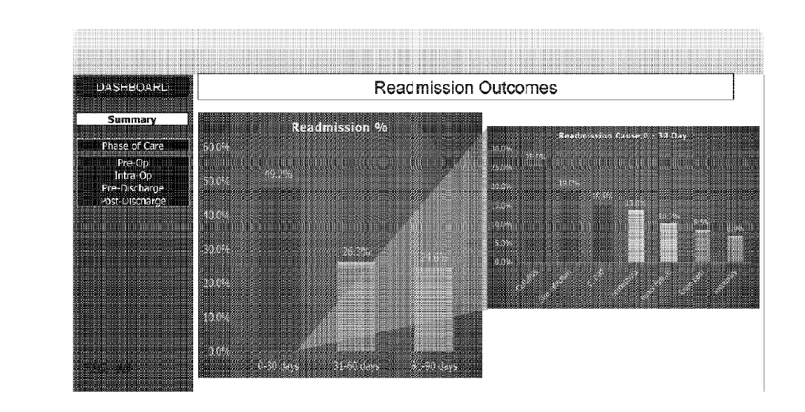

Referring now to FIGS. 4A and 4B, examples of aggregating population based risk for a medical provider (e.g., a single practitioner, a practice group, a clinic, a hospital, a network of providers, etc.) are shown. FIG. 4A shows the monthly and cumulative case volume for a medical provider, which can be obtained by analyzing EMRs. FIG. 4A also shows the 90 day readmission risk, which can be obtained by analyzing a population health model (e.g., a CMS readmission model) with respect to one or more patients. FIG. 4A also shows the 1 year complication risk, which can be obtained by analyzing a population health model (e.g., a CMS complication model) with respect to one or more patients. It should be understood that specific population health models are noted only as examples and that other models can be analyzed to obtain population based risks for a medical provider. FIG. 4A also illustrates the sequential integration of data. FIG. 4B shows readmissions, as well as cause of readmissions, which can be obtained by analyzing EMRs. It should be understood that the information illustrated by FIGS. 4A and 4B can be displayed on a display device such as the display of a client computer 200 of FIGS. 1 and 2.

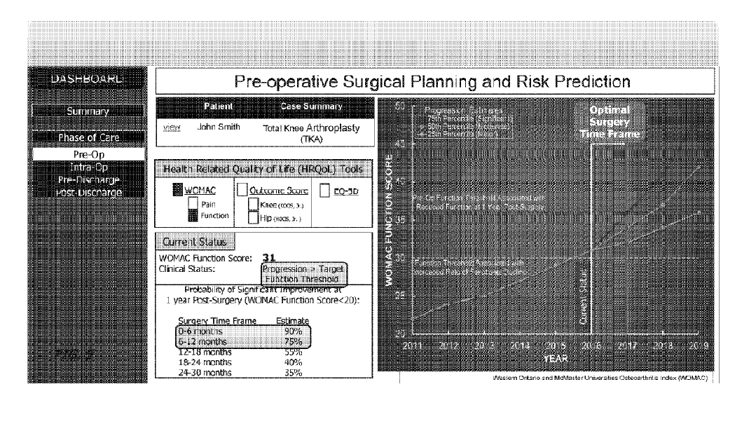

Referring now to FIG. 5, an example of forming a relationship between disease progression and predicting appropriate timing of surgery is shown. FIG. 5 illustrates adjusting functional models (e.g., WOMAC, EQ-5D, Pain VAS, KOOS, HOOS) based on clinical data, which can be obtained by analyzing EMRs, for example. The clinical data can be specific to a patient. The models can be used to predict WOMAC functional scores, pain scores, quality of life, timing of surgery, etc. Optionally, natural progression (e.g., increases/decreases in function, pain, quality of life, etc.) of a condition, which is represented by the model, can be adjusted based on actual clinical data. The future progression (e.g., as shown by the model) can be estimated for one or more rates of change into the future (e.g., beyond current status in 2016). For example, FIG. 5 shows the future progression of functionality for three rates of decline—significant, moderate, and minor. Additionally, optimal intervention timing (e.g., surgical timing) can be predicted from the model as shown in FIG. 5. It should be understood that the information illustrated by FIG. 5 can be displayed on a display device such as the display device of a client computer 200 of FIGS. 1 and 2.

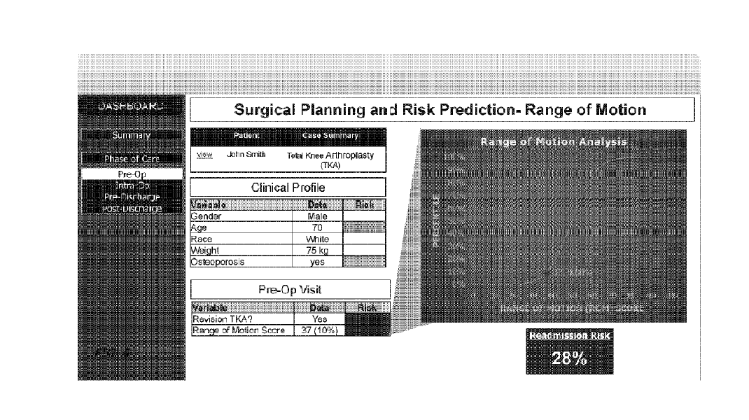

Referring now to FIG. 6, an example of obtaining data in an automated fashion and synthesis of the data creating a unique synthetic profile of the patient is shown. FIG. 6 illustrates obtaining a unique synthetic metric (e.g., risk admission of 28%) for a patient. The unique synthetic metric can represent a risk of readmission, complication, revision, or other risk. The unique synthetic metric can represent a synthesis of a plurality of hazards ratios for a patient, each hazard ratio being based on a different risk factor. For example, when there are different hazards ratios for different risk factors such as age, osteoporosis, range of motion, etc., the unique synthetic metric can provide a single risk score that is specific to the patient. It should be understood that the information illustrated by FIG. 6 can be displayed on a display device such as the display device of a client computer 200 of FIGS. 1 and 2.

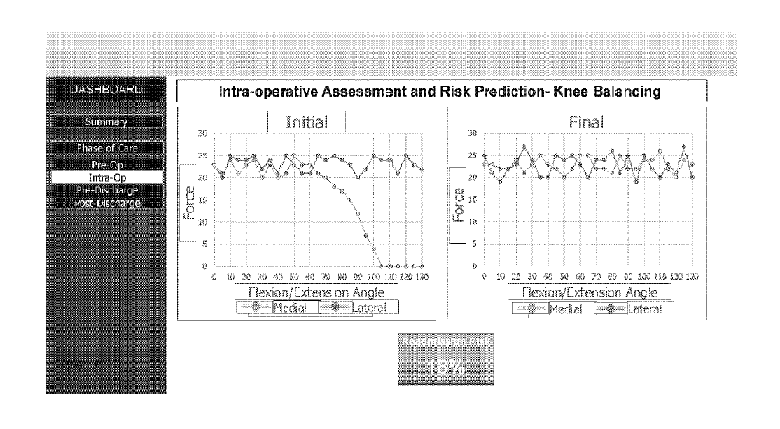

Referring now to FIG. 7, an example of inputting patient-specific parameters obtained during surgery (i.e., intra-operative) using smart implants and/or surgical tools into a predictive model is shown. Examples of smart implants and/or surgical tools are described above and can measure patient-specific parameters during surgery such as force, orientation, temperature, range of motion, or other parameter. This disclosure contemplates measuring and using other patient-specific parameters other than those provided as examples above. Alternatively or additionally, it should also be understood that patient-specific parameters can be obtained from EMRs or other clinical data. This disclosure contemplates that patient-specific parameters can be used with models predicting a risk of readmission, complication, revision, or other risk. In other words, predictive models can be individualized for a particular patient by introducing patient-specific parameters into the models. For example, a patient-specific parameter such as flexion/extension angle can be used with a model to predict the risk of readmission as shown in FIG. 7. Flexion/extension angle is provided only as an example of the patient-specific parameter. This disclosure contemplates using other patient-specific parameters with various predictive models. It should be understood that the information illustrated by FIG. 7 can be displayed on a display device such as the display device of a client computer 200 of FIGS. 1 and 2.

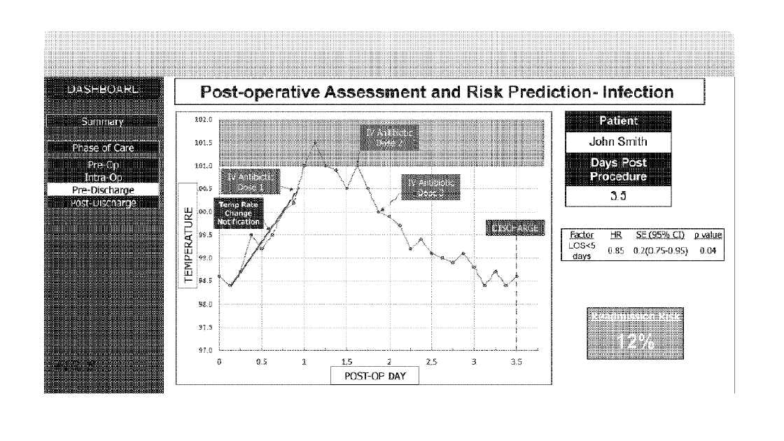

Referring now to FIG. 8, an example of post-operative assessment and risk assessment using patient-specific parameters obtained using smart implants and/or surgical tools is shown. Examples of smart implants and/or surgical tools are described above and can measure patient-specific parameters post-surgery such as force, orientation, temperature, range of motion, or other parameter. This disclosure contemplates measuring and using other patient-specific parameters other than those provided as examples above. Alternatively or additionally, it should also be understood that patient-specific parameters can be obtained from EMRs or other clinical data. This disclosure contemplates that patient-specific parameters can be used with models predicting a risk of readmission, complication, revision, or other risk. In other words, predictive models can be individualized for a particular patient by introducing patient-specific parameters into the models. For example, a patient-specific parameter such as temperature can be used with a model to predict the risk of readmission as shown in FIG. 8. Additionally, the patient-specific parameter(s) can be tracked in real-time, which facilitates timely intervention. In FIG. 8, a large change in temperature is detected during day 1, for example using a smart implant, and a first antibiotic dose is administered quickly, which results in reducing infection risk. Temperature is provided only as an example of the patient-specific parameter. This disclosure contemplates using other patient-specific parameters with various predictive models. It should be understood that the information illustrated by FIG. 8 can be displayed on a display device such as the display device of a client computer 200 of FIGS. 1 and 2.

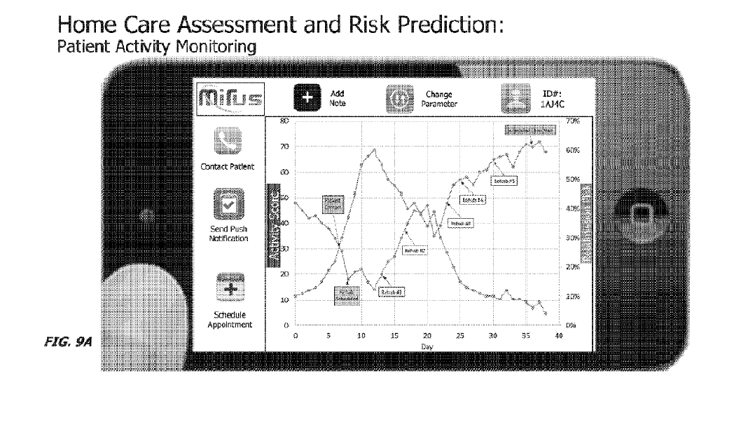

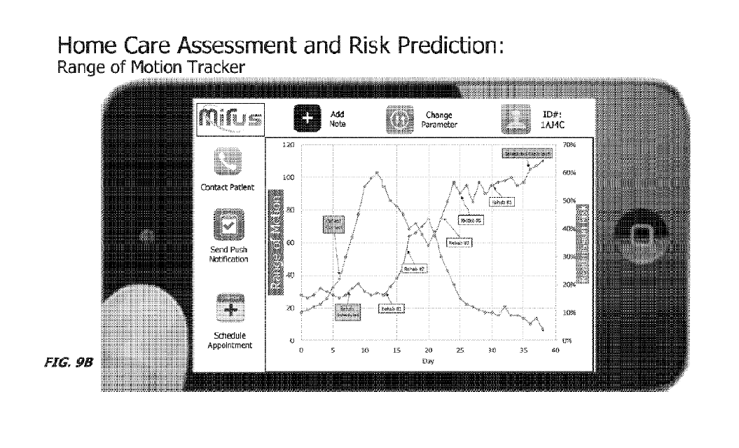

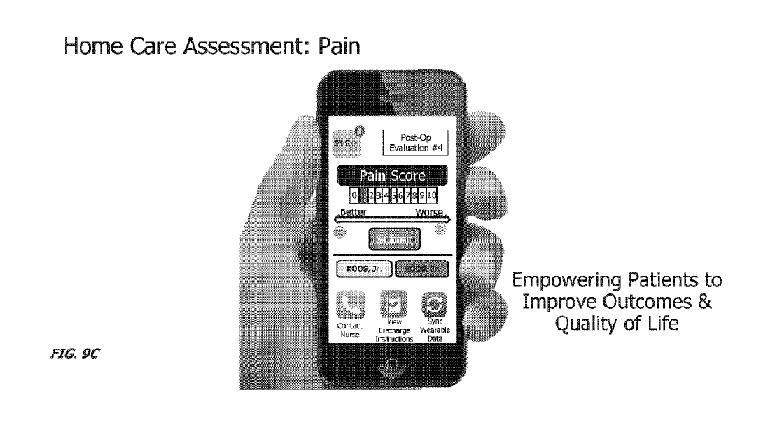

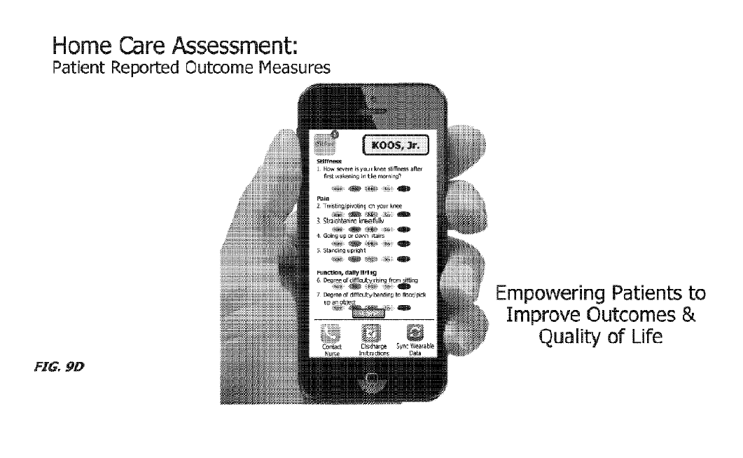

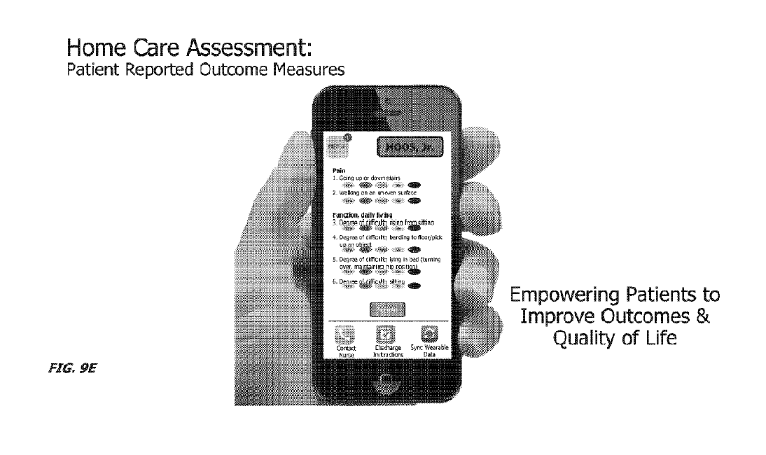

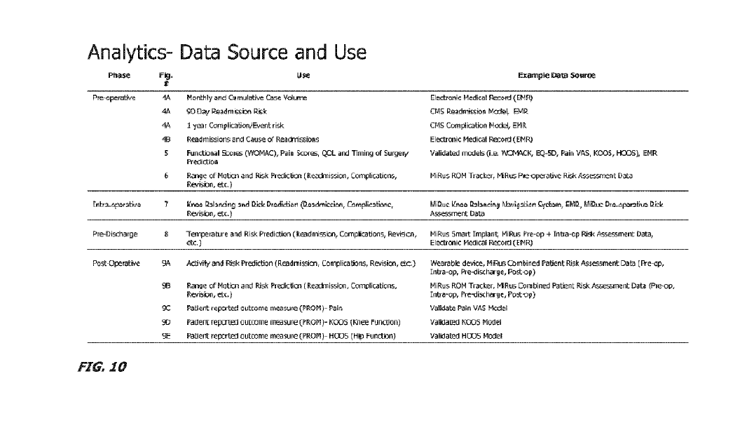

Referring now to FIGS. 9A-9E, examples of providing real-time information from patient to a medical provider are shown. FIG. 9A shows home care assessment and risk prediction (e.g., risk of readmission, complication, revision, or other risk). By providing a wearable device to a patient, patient activity can be monitored and provided to a medical provider in real-time as shown in FIG. 9A. This disclosure contemplates that the information collected by the wearable device can be transmitted to the medical provider (e.g., to servers 100 shown in FIGS. 1 and 2) over a network using an application running on a client device (e.g., client device 200 shown in FIGS. 1 and 2). FIG. 9B shows home care assessment and risk prediction (e.g., risk of readmission, complication, revision, or other risk). By providing a wearable device to a patient, patient range of motion can be monitored and provided to a medical provider in real-time as shown in FIG. 9B. This disclosure contemplates that the information collected by the wearable device can be transmitted to the medical provider (e.g., to servers 100 shown in FIGS. 1 and 2) over a network using an application running on a client device (e.g., client device 200 shown in FIGS. 1 and 2). FIG. 9C shows home care assessment of pain. By providing an interface to report pain (e.g., via an application running on a client device), patient pain level can be monitored and provided to a medical provider in real-time as shown in FIG. 9C. This disclosure contemplates that the information collected at the interface can be transmitted to the medical provider (e.g., to servers 100 shown in FIGS. 1 and 2) over a network using an application running on a client device (e.g., client device 200 shown in FIGS. 1 and 2). FIGS. 9D and 9E show home care assessment of joint function—knee in FIG. 9D and hip in FIG. 9E. By providing an interface to report joint function (e.g., via an application running on a client device), patient joint function can be monitored and provided to a medical provider in real-time as shown in FIGS. 9D and 9E. This disclosure contemplates that the information collected at the interface can be transmitted to the medical provider (e.g., to servers 100 shown in FIGS. 1 and 2) over a network using an application running on a client device (e.g., client device 200 shown in FIGS. 1 and 2). Additionally, this disclosure contemplates that patient-specific information collected as shown in FIGS. 9A-9E can be used with models predicting a risk of readmission, complication, revision, or other risk. It should be understood that the information illustrated by FIGS. 9A-9E can be displayed on a display device such as the display device of a client computer 200 of FIGS. 1 and 2.

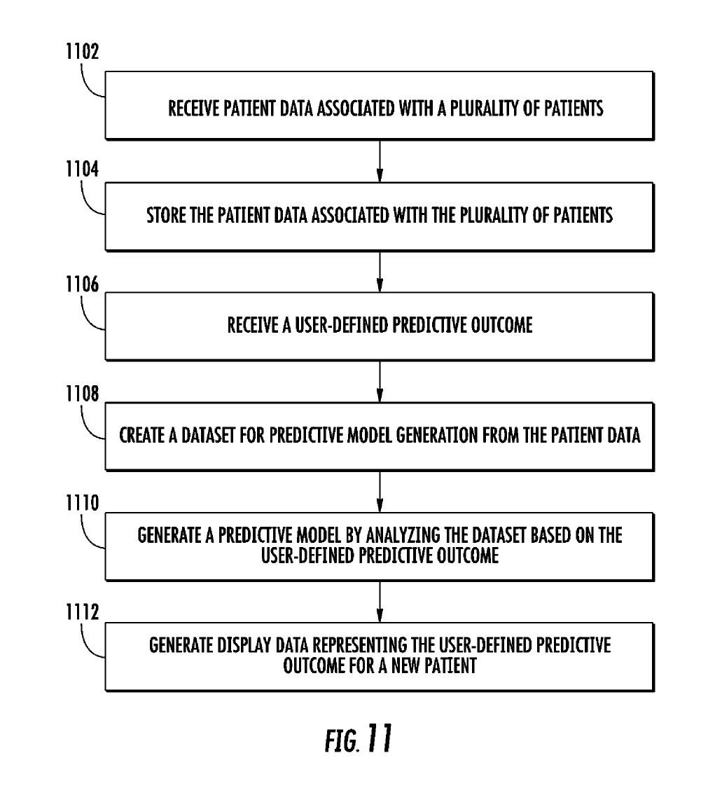

Referring now to FIG. 11, a flow chart illustrating example operations for patient-specific predictive modelling is shown. At 1102, patient data associated with a plurality of patients can be received. This disclosure contemplates that the patient data can be received by a server (e.g., server 100 shown in FIGS. 1 and 2) over a network. As described herein, the patient data can come from various sources and can include, but is not limited to, medical history, social history, comorbidities, demographic information, lab results, vital signs, wearable data, patient-reported outcomes, pain measures, functional measures, quality of life measures, and billing data. In some implementations, the patient data is received at the server using an application program interface (API) configured to interface with respective electronic medical records (EMRs) associated with the plurality of patients. As described above, any API known in the art configured to provide access to and/or retrieve data from EMRs can be used with the methods described herein. Alternatively or additionally, in some implementations, the patient data is received at the server via respective applications running on respective client devices (e.g., mobile or smartphone applications running on a client device shown in FIGS. 1 and 2) associated with the plurality of patients. For example, patient data can be collected from patients in real time as described with regard to FIGS. 9A-9E. Alternatively or additionally, in some implementations, the patient data is received at the server via a navigation system, a wearable device, a smart implant, or a smart surgical tool. Example navigation systems, smart implants, and/or surgical tools that can be used with the methods described herein are provided above. At 1104, the patient data can be stored, for example, in memory (including removable or non-removable storage) accessible to the server.

At 1106, a user-defined predictive outcome can be received at the server. The user-defined predictive outcome can be any outcome that a medical provider such as a doctor, physician, surgeon, nurse, or other medical professional would like to predict. Optionally, the user-defined predictive outcome can be chosen at a client device (e.g., client device 200 shown in FIGS. 1 and 2) and transmitted to the server over the network. For example, a predictive outcome can be categorical (e.g., experiencing a 30-day readmission versus no 30-day readmission), continuous (e.g., post-operative functional index score), a continuous measure that is converted to categorical based upon clinical judgement (e.g., post-operative functional index score greater than or equal to 10 point improvement versus post-operative functional index score less than 10 point improvement), time-dependent outcomes that are limited to a single occurrence (e.g., time to death), time-dependent outcomes that can have multiple occurrences (e.g., hospitalization rates). It should be understood that the predictive outcomes provided above are only examples and that other user-defined predictive outcomes can be used with the methods described herein.

At 1108, a dataset for predictive model generation can be created from the patient data. As described below, this can include creating and appending one or more output vectors to elements of the patient data. The user-defined predictive outcome can be applied to the available patient data to derive a working dataset for model generation. In other words, the patient data stored by and/or accessible to the server can be screened against the user-defined predictive outcome. For example, the user-defined predictive outcome can be 30-day readmission to a hospital in an example implementation. While screening the patient data, an outcome vector of value 1 can be created if a given patient experienced a hospitalization within 30 days of the discharge date of index hospitalization, and an outcome vector of value 0 can be created if a given patient did not experience hospitalization within 30 days of the discharge date of index hospitalization. In this example, the outcome vector can be derived by evaluating the respective medical histories for a plurality of patients, which can be obtained from the EMRs as described herein. The outcome vector can be appended to the data input matrix (e.g., a data element within the patient data) resulting in a working dataset.

At 1110, a predictive model can be generated by analyzing the dataset based on the user-defined predictive outcome. As described below, this step can include performing a statistical analysis of the patient data. Based upon the user-defined predictive outcome received in step 1106 and the dataset generated in step 1108, a statistical regression technique can be applied to fit a set of independent predictor variables (e.g., elements contained in the patient data received at step 1102). Statistical regression techniques are known in the art and are therefore not described in further detail below. Examples include logistic regression for binary and ordinal defined outcomes, linear regression/multiple linear regression for continuous defined outcomes, cox proportional hazards regression for time-dependent single event outcomes, Andersen-gill extension of the cox proportional hazards regression for time dependent multiple event outcomes, and other generalized linear model (GLM) techniques. It should be understood that the statistical analyses provided above are only examples and that other statistical analyses can be used with the methods described herein. This disclosure contemplates that predictor variables can enter into the model automatically based upon clinical judgement and/or be added/removed through established statistical techniques (e.g. stepwise, backward elimination). In addition, model fit parameters (e.g., Akaike information criterion (AIC), c-statistics, etc.) can optionally be obtained, which provide information regarding the quality of the predictive model. The predictive model can be applied to a new patient, for example, a new patient that was not part of the dataset generated at step 1108 (i.e., the historical dataset). The outcome of interest (e.g., the user-defined predictive outcome) can then be estimated for this new patient. Alternatively or additionally, in some implementations, an actual outcome associated with this new patient (e.g., whether or not the new patient experienced readmission to a hospital within 30 days) can be received, and the patient data (i.e., the historical dataset) can be updated accordingly to include this information. For example, the new patient to which the predictive model was applied will obtain an outcome output value (e.g., the new patient either experiences a 30-day readmission or does not). At this point, the new patient and his/her outcome value can be added to the historical dataset. The predictive model can thereafter be regenerated. Optionally, model fit parameters can be obtained and compared with the original model to determine model fit improvement.

At 1112, display data can be generated. The display data can be transmitted to a client device (e.g., client device 200 shown in FIGS. 1 and 2) over the network. The display data can represent the user-defined predictive outcome for the new patient. In some implementations, the display data can be a binary outcome plotted as a function of a continuous variable. For example, the display data can be a binary outcome such as probability of 30-day hospital readmission (or complication, revision, other risk, etc.) plotted as a function of time from discharge, activity level (e.g., based on analysis of wearable data), age, etc. It should be understood that the binary outcomes and/or continuous variables provided above are only examples and that other binary outcomes and/or continuous variables can be used with the methods described herein. The display data can be displayed, for example, at a graphical user interface (GUI) of a client device (e.g., client device 200 shown in FIGS. 1 and 2). This type of graphical display can be helpful to a clinician (e.g., doctor, physician, surgeon, or other medical professional) and/or a patient in guiding treatment, rehabilitation, or recommendation for surgical intervention.

### Examples

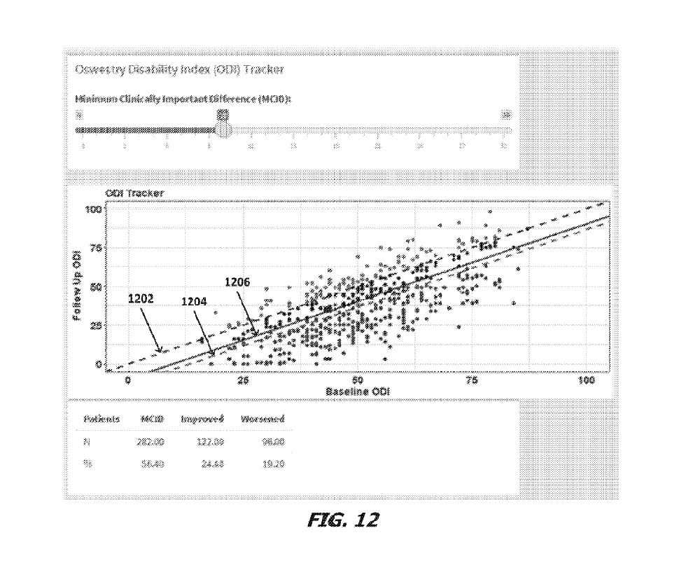

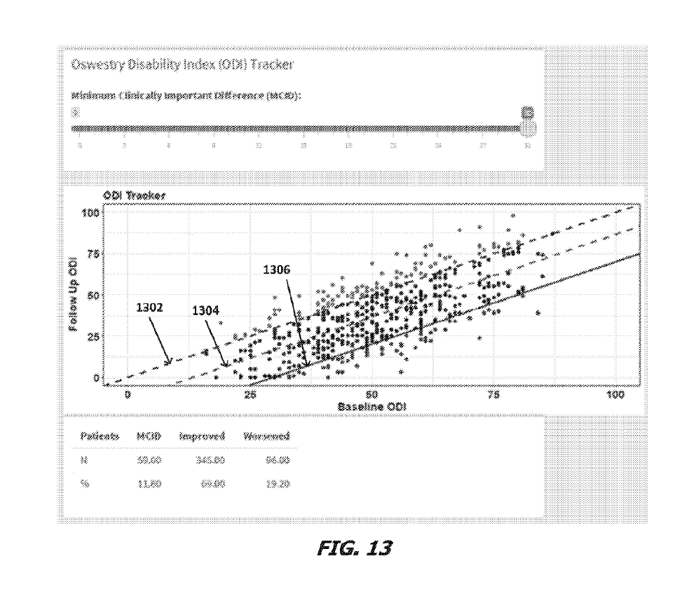

Referring now to FIGS. 12 and 13, example graphs illustrating follow up versus baseline Oswestry Disability Index (ODI) scores are shown. ODI is an index used to quantify disability for lower back pain. ODI is known in the art and is not described in further detail below. Tracking ODI scores over time (e.g., changes from baseline to follow up) provides a measure of disability progression/regression. It should be understood ODI is provided only as an example and that other metrics (e.g., other disability indexes) can be used with the methods described herein. Patient data for a plurality of patients can be aggregated at a server (e.g., server 100 shown in FIGS. 1 and 2) to create a database. An example of this process is described above with regard to Steps 1102 and 1104 shown in FIG. 11. The patient data can include ODI values for a plurality of patients. The ODI values can be obtained from the patients' respective EMRs, for example, as described herein. This historical dataset (n=500) can be used to create graphs such as those shown in FIGS. 12 and 13.

A user can visualize the relationship between Baseline ODI scores and Follow Up ODI scores for the historical patient dataset (n=500) by examining FIGS. 12 and 13. In FIG. 12, dashed line 1202 (y=x) separates those patients that worsened (i.e., follow up ODI score>baseline ODI score) from those patients that improved (i.e., follow up ODI score≤baseline ODI score). Additionally, in FIG. 12, dashed line 1204 plots the average difference in follow up ODI score for the historical dataset. Further, a user can define the minimum clinically important difference (MCID) threshold, which is shown by solid line 1206 in FIG. 12. The MCID threshold in FIG. 12 is set at 10 (i.e., follow up ODI score is at least 10 points less than baseline ODI score). The MCID threshold is shown by the slide bar ODI tracker of FIG. 12. It should be understood that an MCID threshold of 10 is provided only as an example and that it can have other values. Line 1206 separates or distinguishes those patients that simply improved (i.e., dots found between lines 1202 and 1206) from those patients that met the MCID threshold (i.e., dots found below line 1206). Patients that worsened are represented by dots found above line 1202. As shown in FIG. 12, 56.40% (N=282) of patients met the MCID threshold (i.e., follow up ODI score at least 10 points less than baseline ODI score), while another 24.40% (N=122) of patients saw improvement (i.e., follow up ODI score less than baseline ODI score but difference does not exceed 10 points). On the other hand, 19.20% (N=96) of patients worsened (i.e., follow up ODI score>baseline ODI score).

In FIG. 13, the MCID threshold is changed to 30. Optionally, this can be accomplished by user adjusting he slide bar ODI tracker of FIG. 13, which can be displayed on a client device (e.g., client device 200 shown in FIGS. 1 and 2). The historical dataset (n=500) is the same as that shown in FIG. 12. In FIG. 13, dashed line 1302 (y=x) separates those patients that worsened (i.e., follow up ODI score >baseline ODI score) from those patients that improved (i.e., follow up ODI score≤baseline ODI score), dashed line 1304 plots the average difference in follow up ODI score for the historical dataset, and the minimum clinically important difference (MCID) threshold is shown by solid line 1306. Line 1306 separates or distinguishes those patients that simply improved (i.e., dots found between lines 1302 and 1306) from those patients that met the MCID threshold (i.e., dots found below line 1306). Patients that worsened are represented by dots found above line 1302. As shown in FIG. 13, only 11.80% (N=59) of patients met the MCID threshold (i.e., follow up ODI score at least 30 points less than baseline ODI score), while another 69.00% (N=345) of patients saw improvement (i.e., follow up ODI score less than baseline ODI score but difference does not exceed 30 points). On the other hand, 19.20% (N=96) of patients worsened (i.e., follow up ODI score>baseline ODI score).

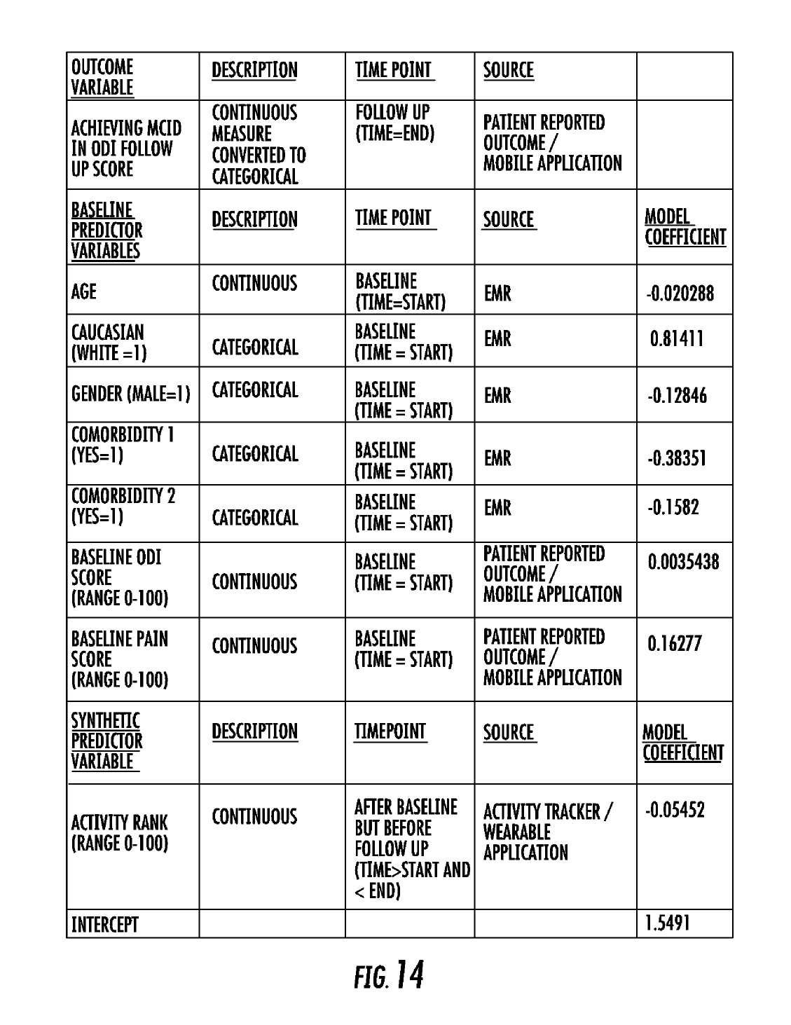

Referring now to FIG. 14, an example table illustrating dataset generation is shown. An example of this process is described above with regard to Steps 1106 and 1108 shown in FIG. 11. For example, a user can define an outcome of interest (e.g., a user-defined predictive outcome) for model fitting based on those patients that achieved MCID threshold at follow up. In FIG. 14, the outcome of interest (i.e., outcome variable in the table) is the binary outcome of achieving the MCID threshold (e.g., MCID threshold=10 in FIG. 12 or MCID threshold=30 in FIG. 13) in follow up ODI score as compared to baseline ODI score. In FIG. 14, the MCID (or change in ODI score between follow up and baseline) is converted from a continuous measure to a binary outcome (i.e., categorical) based on whether the change in ODI score between follow up and baseline meets the MCID threshold. An output vector can be appended to the patient dataset that distinguishes those patients that met the MCID threshold (e.g. value=1) from those that did not (e.g. value=0). This process is described above with regard to Step 1108 shown in FIG. 11. The table in FIG. 14 illustrates example baseline predictor variable values, as well as the time and source of such information. The example baseline predictor variable values include age (e.g., continuous value), ethnicity (e.g., value=1 Caucasian; value=0 all other ethnicities), gender (e.g., value=1 male; value=0 female), one or more comorbidities such as a disease or condition (e.g., value=1 present; value=0 not present), baseline ODI score (e.g., continuous value), and baseline pain score (e.g., continuous value). It should be understood that the baseline predictor variables (and their respective values) provided above are only examples and that more, less, and/or other predictor variables (and their values) can be used. The predictor variables can be found in the patient data, which comes from various sources as described herein. The table in FIG. 14 also illustrates example model coefficients associated with the baseline predictor variables.

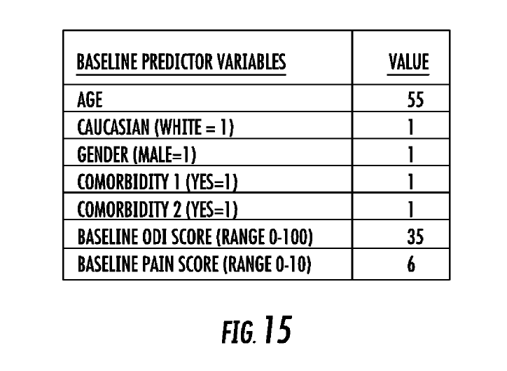

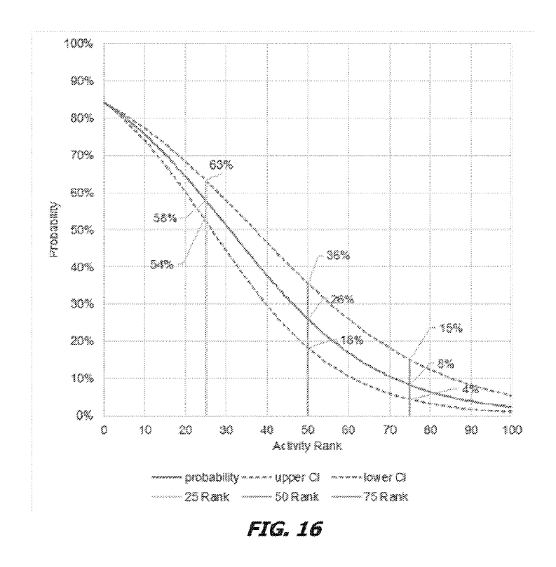

Referring now to FIG. 15, an example table illustrating baseline predictor variable values for a new patient (Patient A) are shown. A predictive model can then be generated. This process is described above with regard to Step 1110 shown in FIG. 11. For example, a statistical analysis can be performed on the dataset to generate a model that predicts the outcome defined by the user. In this example, the model coefficients (i.e., shown in the table of FIG. 14) that estimate the probability of achieving MCID threshold in follow up ODI score (user defined outcome) can be applied to Patient A by regressing the model coefficients with the patient-specific baseline predictor variable values (i.e., shown in the table of FIG. 15). For example, the probability of Patient A achieving MCID threshold in follow up ODI score can be then regressed as a function of Activity Rank. It should be understood that Activity Rank is one example synthetic predictor variable (e.g., a unique synthetic metric described above with regard to FIG. 6). This disclosure contemplates that the synthetic predictor variable is not limited to Activity Rank and can be other synthetic metrics. Additionally, Activity Rank can serve as the continuous variable against which the binary outcome (e.g., patient meeting MCID threshold) is plotted. The example result is shown in FIG. 16, which is a graph illustrating Patient A's probability of meeting an MCID threshold as a function of Activity Rank. In other words, FIG. 16 is an example of display data representing the user-defined predictive outcome for a new patient, where the display data is the probability of a binary outcome plotted as a function of a continuous variable. This process is described above with regard to Step 1112 shown in FIG. 11. Using FIG. 16, a user (e.g., medical professional or patient) can tailor an activity regimen for the patient to target after Baseline measurement but before the end of a Follow Up period that is associated with a desired probability. For example, if the user would like to ensure Patient A has a probability of achieving the MCID threshold in follow up ODI score equal to ˜50% or more, Patient A should target an activity regimen that would rank them in the top 25 percent (Rank 25).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method, comprising:
receiving, at a server, patient data over a network, the patient data being associated with a plurality of patients;
storing, in memory accessible by the server, the patient data;
receiving, at the server, a user-defined predictive outcome over the network;
creating, using the server, a dataset for predictive model generation from the patient data;
generating, using the server, a predictive model by analyzing the dataset based on the user-defined predictive outcome; and
generating, using the server, display data representing the user-defined predictive outcome for a new patient.

2. The method of claim 1, wherein the display data representing the user-defined predictive outcome for the new patient comprises a binary outcome plotted as a function of a continuous variable.

3. The method of claim 1 or 2, further comprising displaying, at a graphical user interface (GUI) of a client device, the display data representing the user-defined predictive outcome for the new patient.

4. The method of any one of claims 1-3, wherein the patient data is received at the server using an application program interface (API) configured to interface with respective electronic medical records (EMRs) associated with the plurality of patients.

5. The method of any one of claims 1-4, wherein the patient data is received at the server via respective applications running on respective client devices associated with the plurality of patients.

6. The method of any one of claims 1-5, wherein the patient data is received at the server via a navigation system, a wearable device, a smart implant, or a smart surgical tool.

7. The method of any one of claims 1-6, wherein creating the dataset for predictive model generation from the patient data using the server comprises creating and appending one or more output vectors to elements of the patient data.

8. The method of any one of claims 1-7, wherein analyzing the dataset based on the user-defined predictive outcome comprises performing a statistical analysis of the patient data.

9. The method of claim 8, wherein the statistical analysis is at least one of a logistic regression, a linear regression, a proportional hazards regression, or a generalized linear model (GLM).

10. The method of any one of claims 1-9, further comprising:
receiving, at the server, an actual outcome associated with the new patient; and
updating, using the server, the patient data to include the actual outcome associated with the new patient.

11. The method of claim 10, further comprising regenerating, using the server, the predictive model.

12. A system, comprising:
one or more client devices; and
a server communicatively connected to the one or more client devices over a network, the server having a processor and a memory operably coupled to the processor, wherein the memory has computer-executable instructions stored thereon that, when executed by the processor, cause the processor to:
receive patient data over the network, the patient data being associated with a plurality of patients, wherein the patient data is received: using an application program interface (API) configured to interface with respective electronic medical records (EMRs) associated with the plurality of patients; via respective applications running on the one or more client devices; or via a navigation system, a wearable device, a smart implant, or a smart surgical tool,
store, in the memory, the patient data,
receive a user-defined predictive outcome over the network,
create a dataset for predictive model generation from the patient data,
generate a predictive model by analyzing the dataset based on the user-defined predictive outcome, and
transmit display data to at least one of the one or more client devices over the network, the display data representing the user-defined predictive outcome for a new patient.

13. The system of claim 12, wherein the display data representing the user-defined predictive outcome for the new patient comprises a binary outcome plotted as a function of a continuous variable.

14. The system of claim 12 or 13, wherein the display data representing the user-defined predictive outcome for the new patient is displayed at a graphical user interface (GUI) of the at least one of the one or more client devices.

15. The system of any one of claims 12-14, wherein creating the dataset for predictive model generation from the patient data using the server comprises creating and appending one or more output vectors to elements of the patient data.

16. The system of any one of claims 12-15, wherein analyzing the dataset based on the user-defined predictive outcome comprises performing a statistical analysis of the patient data.

17. The system of claim 16, wherein the statistical analysis is at least one of a logistic regression, a linear regression, a proportional hazards regression, or a generalized linear model (GLM).

18. The system of any one of claims 12-17, wherein the memory has further computer-executable instructions stored thereon that, when executed by the processor, cause the processor to:
receive an actual outcome associated with the new patient; and
update the patient data to include the actual outcome associated with the new patient.

19. The system of claim 18, wherein the memory has further computer-executable instructions stored thereon that, when executed by the processor, cause the processor to regenerate the predictive model.

20. A non-transitory computer-readable recording medium having computer-executable instructions stored thereon that, when executed by a processor, cause the processor to:
receive patient data associated with a plurality of patients over a network;
store the patient data;
receive a user-defined predictive outcome over the network;
create a dataset for predictive model generation from the patient data;
generate a predictive model by analyzing the dataset based on the user-defined predictive outcome; and
generate display data representing the user-defined predictive outcome for a new patient.

21. A method, comprising:
receiving, using an application program interface (API), clinical data from an electronic medical record; and
using the clinical data, creating a risk based model for clinical planning or management.

22. A method, comprising:
receiving a patient-specific parameter from a navigation system, a wearable device, a smart implant, or a smart surgical tool; and
using the patient-specific parameter, creating or updating a risk based model for clinical planning or management.

23. The method of claim 21 or claim 22, further comprising generating a patient-specific risk metric using the risk based model.

24. The method of claim 23, wherein the patient-specific risk metric comprises a unique synthetic risk metric based on a plurality of risk factors.

25. The method of claim 23, wherein the patient-specific risk metric comprises a unique synthetic risk metric based on a customized set of risk factors.

26. The method of any one of claims 21-25, wherein the patient-specific risk metric comprises a risk of readmission, complication, or revision.

27. The method of claim 21 or claim 22, wherein the risk based model comprises a progression of a condition or risk over time.

28. The method of claim 27, further comprising estimating an optimal time for an intervention based on the risk based model.

29. The method of claim 22, wherein the patient-specific parameter comprises at least one of force, orientation, position, temperature, wear, loosening, range of motion, or combinations thereof.

30. The method of any one of claims 21-29, further comprising displaying the risk based model on a display device of a computing device.

31. A method, comprising:
aggregating population based risk for a medical provider from a plurality of data sources; and
displaying the population based risk on a display device of a computing device.
